(19) United States
(12) Patent Application Publication
(10) Pub. No.: US 2008/0051692 A1
(43) Pub. Date: Feb. 28, 2008

(54) APPLICATOR CARTRIDGE FOR AN ELECTROKINETIC DELIVERY SYSTEM FOR SELF ADMINISTRATION OF MEDICAMENTS

(75) Inventors: John S. Petersen, Acton, MA (US); Dennis I. Goldberg, Sudbury, MA (US); James W. Kelland, East Walpole, MA (US); Robert W. Ehteredge, Natick, MA (US); Michael S. Barsness, Oxford, MA (US); Kuowei Chang, Lexington, MA (US)

Correspondence Address: NIXON & VANDERHYE, PC, 901 NORTH GLEBE ROAD, 11TH FLOOR, ARLINGTON, VA, 22203, US

(73) Assignee: TRANSPORT PHARMACEUTICALS, INC., Framingham, MA (US)

(21) Appl. No.: 11/565,335

(22) Filed: Nov. 30, 2006

Related U.S. Application Data
(60) Provisional application No. 60/740,678, filed on Nov. 30, 2005.
(60) Provisional application No. 60/743,528, filed on Mar. 17, 2006.
(60) Provisional application No. 60/743,746, filed on Mar. 24, 2006.

(51) Int. Cl. A61N 1/30

(52) U.S. Cl. 604/20

(57) ABSTRACT

An applicator for a device for electrokinetic delivery of medicament to a treatment site includes an applicator head having an active electrode, a matrix, and a medicament or a medicament and an electrically conductive carrier, carried by the matrix in electrical contact with the electrode. The cartridge includes a plurality of raised projections allowing the matrix and head to be ultrasonically welded one to the other. The electrode opens through a face of the head remote from the matrix for connection with an electrical connector carried by the device. A lid overlies the matrix and is releasably attached to a margin of the head. The lid includes layers of different materials with one of the layers formed of a metallic material having a discontinued interface with the lid and a tab carried by the lid.

### BACKGROUND OF INVENTION

This patent application is related to, and claims the benefit of, U.S. Provisional Patent Application No. 60/740,678, filed Nov. 30, 2005, and 60/743,528, filed Mar. 17, 2006, the entire disclosures of these provisional applications are incorporated herein by reference. This application is also related to concurrently filed U.S. patent application Ser. No. ______, (NV Ref. 3589-78) and entitled “Combination Cartridge And Device For Electrokinetic Delivery Of Medicament To A Treatment Site,” the entire disclosure of this application is incorporated herein by reference.

The present invention relates generally to the electrokinetic mass transfer of substances to tissue and particularly relates to an apparatus for electrokinetically delivering substances, e.g., a medicament to a treatment site.

Electrokinetic delivery of medicaments for applying medication locally through a human individual's skin to a treatment site is known. One type of electrokinetic delivery mechanism is iontophoresis, i.e., the application of an electric field to the skin to enhance the skin's permeability and to deliver various ionic agents, e.g., ions of salts or other drugs to the treatment site. In certain situations, iontophoretic or transdermal or transmucosal cutaneous delivery techniques have obviated the need for hypodermic injection of many medicaments thereby eliminating the concomitant problem of trauma, pain and risk of infection to the individual. Other types of electrokinetic delivery mechanisms include electroosmosis, electroporation, and electromigration, any or all of which are more generally known as electrotransport, electromolecular transport or iontophoretic methods, all of which are collectively known as electrokinetic methods.

Prior electrokinetic devices for delivering medicaments to a treatment site were cumbersome, bulky and costly and oftentimes required the presence of an individual at a doctor's office or treatment center and use of medical professionals to administer the medicament. More recently, devices have been developed for the private self administration of medicaments or for diagnostic application by the individual at non-medical or non-professional facilities. For example, in U.S. Pat. No. 6,792,306, there is disclosed an electrokinetic delivery device which includes a housing containing a power source, electronics and a counter electrode, the device being shaped and configured for releasable securement to an individual's finger and terminating in an applicator head having an active electrode. By applying the applicator head to the skin overlying the treatment site and with the medicament or a medicament and a carrier therefor carried by the applicator head, the medicament may be electrokinetically delivered to the treatment site. There has developed a need, for an applicator head which can be readily and easily manufactured at low cost and which protects the medicament whereby the applicator head can be secured to a portable electrokinetic medicament delivery device.

### SUMMARY OF INVENTION

An applicator has been developed for use with a device for electrokinetically delivering a medicament to a treatment site on a human comprising an applicator head including an active electrode and a matrix; the applicator head beyond the active electrode has a plurality of sites for attachment of the matrix; the matrix and the applicator head are attached to one another at one or more of these sites; the active electrode is positioned within or underneath the matrix, and a medicament or a medicament with an electrically conductive carrier carried by the matrix is in electrical contact with the electrode.

An applicator has been developed for use with a device for electrokinetically delivering a medicament to a treatment site comprising an applicator head including an active electrode and a matrix; a medicament or a medicament with an electrically conductive carrier present within the matrix and in electrical contact with the electrode; a lid overlying the matrix on a side of the matrix remote from the electrode and releasably secured to the applicator head; and the lid comprising layers of different materials and including one or more tabs, one of the layers of the lid and the tab being formed of a metallic material. In one embodiment of the lid, at least a portion of an interface between the metallic material of the tab and the metallic material of the lid is discontinuous to facilitate proper induction sealing of the lid while maintaining an unsealed tab for lid removal. In another embodiment, the lid may be an an oversized disc having a rim constituting an annular tab.

An applicator has been developed for use with a device for electrokinetically delivering a medicament to a treatment site comprising an applicator including an active electrode and a matrix; a medicament or a medicament with an electrically conductive carrier is present within the matrix; the cartridge beyond the active electrode and the matrix being secured to one another; the active electrode having a first portion exposed through a first face of 3589-92 the applicator head remote from the matrix; and another portion of the active electrode being exposed to the matrix along a second face of the applicator head for electrical contact with the medicament or the medicament and the electrically conductive carrier therefor.

An applicator has been developed for use with a device for electrokinetically delivering a medicament to a treatment site comprising: an applicator head having a recess, an active electrode in said recess and a matrix in said recess overlying said active electrode; a medicament or a medicament and an electrically conductive carrier carried by said matrix; a margin of said applicator head within said recess, said matrix being secured to the margin, and said active electrode including an electrical contact exposed through a side of said applicator head opposite to the recess.

An applicator device has been developed for electrokinetically delivering a medicament to a treatment site comprising: a housing including a power supply and an electronic circuit providing current for delivering the medicament; a cartridge head having an active electrode and a matrix overlying the active electrode, wherein the active electrode is in electrical communication with power supply and electronic circuit when the head is inserted into the housing; a medicament or a medicament and an electrically conductive carrier carried by said matrix and in electrical contact with said active electrode, and a locking element on the cartridge head and releaseably engaged with the housing, the locking element having a first conductive contact surface abutting a second conductive contact surface in the housing, wherein the first conductive contact surface is in a conductive path with the active electrode and the second conductive contact surface is in a conductive path with the power supply and electronic circuit, and further the abutment of the first conductive surface and the second conductive surface establishes a electrical connection between the surfaces.

### DESCRIPTION OF THE DRAWINGS

FIG. 1 is a perspective view of an exemplary electrokinetic delivery device including an applicator head secured to the delivery device.

FIG. 2 is a view similar to FIG. 1 illustrating the device with one side of the housing removed to show internal components.

FIG. 3 is an exploded view of the applicator head as viewed from its backside.

FIG. 4 is an exploded view of the applicator head illustrating an applicator head lid, matrix and head as viewed from the front side of the head, i.e., the side contacting the individual's skin.

FIG. 5 is a side perspective view of the applicator head.

FIG. 6 is an enlarged cross-sectional view of the applicator head secured to the device.

FIG. 7 is an enlarged cross-sectional view of a lid for the applicator head.

FIG. 8 illustrates an alternative device having a flex circuit and pogo stick connector between the device and an active electrode.

FIG. 9 is a schematic diagram of the printed circuit board in the device and electric components connected to the printed circuit board in the housing of the device.

FIG. 10 is a schematic diagram of electrical contact pads that may be mounted on opposing surfaces on the cartridge head and housing such that the pads are conductively connected with the cartridge head is locked in the housing.

### DETAILED DESCRIPTION OF THE INVENTION

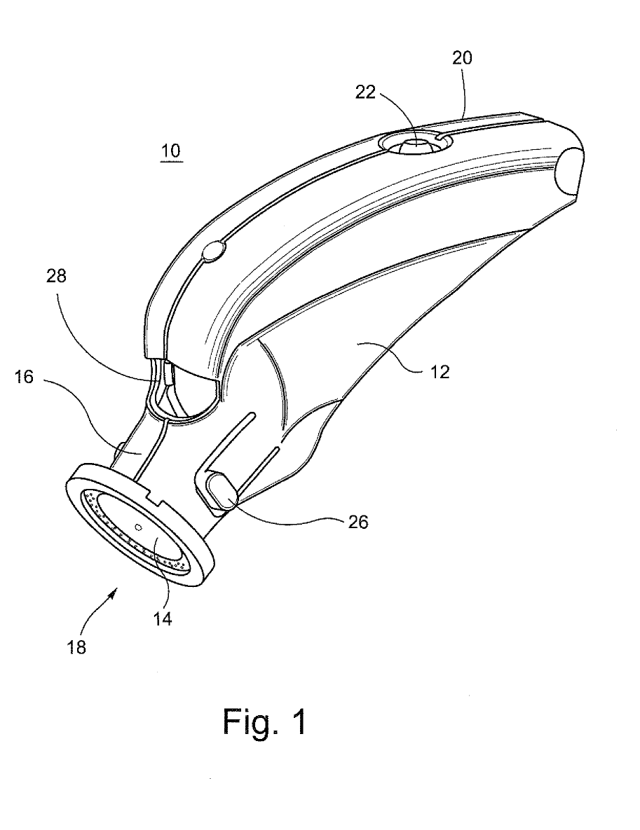

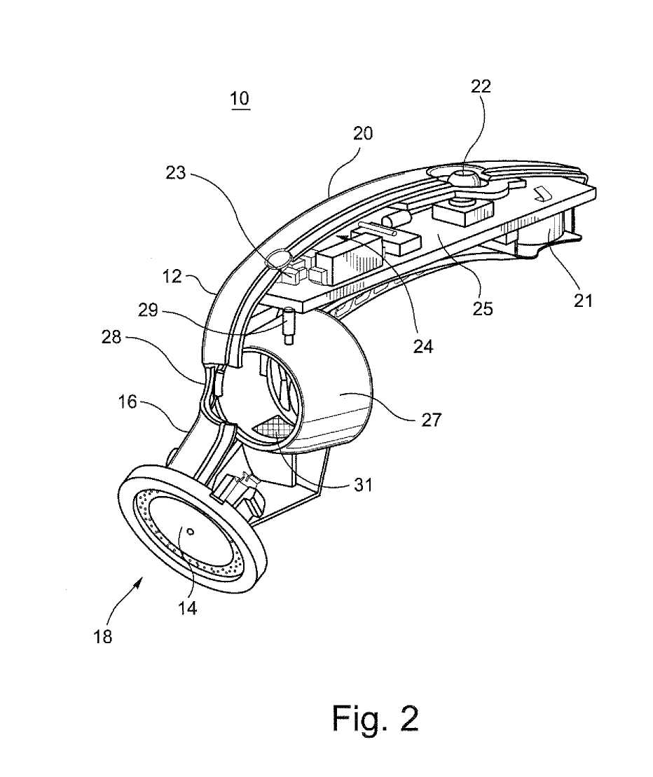

FIGS. 1 and 2 show a portable, self contained, lightweight, compact, finger mounted, electrokinetic medicament delivery device or medicator 10 (collectively a “device”) for application to a treatment site (TS) on the skin of an individual. The device 10 includes a housing 12 mountable to an individual's finger with the distal end 16 of the device 10 mounting an applicator cartridge 18 containing an active electrode. The device 10 drives, e.g., electrokinetically transports, medicament interposed between the active electrode 14 and the individual's treatment site into the treatment site upon completion of an electrical circuit through the device, the active electrode, the medicament or hydration material carrying the medicament (collectively referred to as “medicament”), the individual's body and a counter electrode, i.e., tactile electrode carried by the device. While FIGS. 1 and 2 show the active electrode 14 exposed on the front face of the cartridge 18, the electrode is typically covered by a medicament matrix that is attached to a front face of the cartridge head and applied to the treatment site. The device 10 carrying the applicator cartridge 18 provides a facile and fatigue-free approach to the affected treatment site. The housing, in one embodiment, need not be secured to the finger with straps or other fixtures for immobilizing the finger.

The housing 12 is preferably formed of a plastic material, although other materials may be utilized. A unitary housing or a housing formed of more than one part may be provided. Additionally while the housing is substantially rigid, the ergonomic design of the housing allows freedom of movement of the finger without losing critical contact between the skin and the device. Furthermore, this ergonomic design of the housing permits a bent-finger application of the device to the face reducing finger joint fatigue and enabling greater contact force between the applicator cartridge head and the face via fingertip pressure applied.

The housing 12 may include an internal compartment 24 for receiving a printed circuit board 25 containing a battery power source 21. By example, one or more batteries of various sizes can be accommodated in the compartment 24 of the housing and an external power source may also be used. In one configuration, the device 10 is disposable (after several treatments each with a separate cartridge head) and contains no user-replaceable batteries. The device may be configured with consumer replaceable batteries or a rechargeable power supply in the sealed housing. Alternatively, the power source may be a cord connection to a wall socket power source. The printed circuit board 25 or other electronic package may control current applied to the active electrode, time of current delivery to the active electrode, provide redundant safety features (such as a switch to prevent application of the current without a cartridge head in the device), and ensure user visual and/ or audible signaling during use of the device, e.g., activation of the circuit board LED transmitted through the light pipe 23.

A proximal portion 20 of the housing 12 is elongated and shaped to fit comfortably on the top of the user's index finger. Located on the top surface of the housing is a manually actuated switch button 22 for energizing the circuitry and preparing the device for use. This button 22 may be a separate part, molded as a cantilever feature integral to the housing, or incorporated as part of an overmolded elastomeric part of the housing.

Further down the top side of the device toward the distal end 16 and located in a position that affords maximum visibility is a light pipe 23 that captures and transmits light signals from the circuit board light emitting diodes (LED) to the user. The exposed light pipe 23 is shaped to provide significant forward reflection of the light while maintaining visibility at other viewing angles (such as a reflected image in a mirror). This reduces user fatigue by allowing the user to view device light signals directly at close range or at greater distance in a mirror.

Forward of the light pipe is an opening 28 in the housing that provides an access port through which long fingernails may extend. The opening allows finger nails and long fingers to project through the housing so that good contact may be made between the fleshy pad of the finger and the ring 27. The opening facilitates proper contact with the counter electrode, e.g., contact surface 31, on an inside surface of the ring 27.

Adjacent to either side of the fingernail port 28 are ejector buttons 26 that, when depressed, disengage the applicator and provide a forward movement of the applicator away from the device during disposal. The exact size and position of the ejection and/or release features may be varied in response to the size of the applicator. The ejection and/or release features work in concert with the applicator and prevent inadvertent ejection or release during use.

Within the distal portion 16 of the housing 12 and mechanically captured by the housing is a conductive ring 27 that contacts a portion of an individual's finger, preferably an index finger. The conductive ring 27 provides a secure electrical contact with the wide range of finger sizes, thereby completing the electrical circuit which includes the PCB and the face of the drug cartridge 18. The conductive surface 31 serves as a counter electrode in the circuitry of the device and may be embodied as a pad on an inside surface of the ring 27. The conductive surface 31 is connected to the circuitry of the printed circuit board and provides an electrical connection between the circuit board and the user that is remote to the active electrode and treatment site. The conductive ring may be comprised of metals, metal coatings, conductive polymers, or combinations of these.

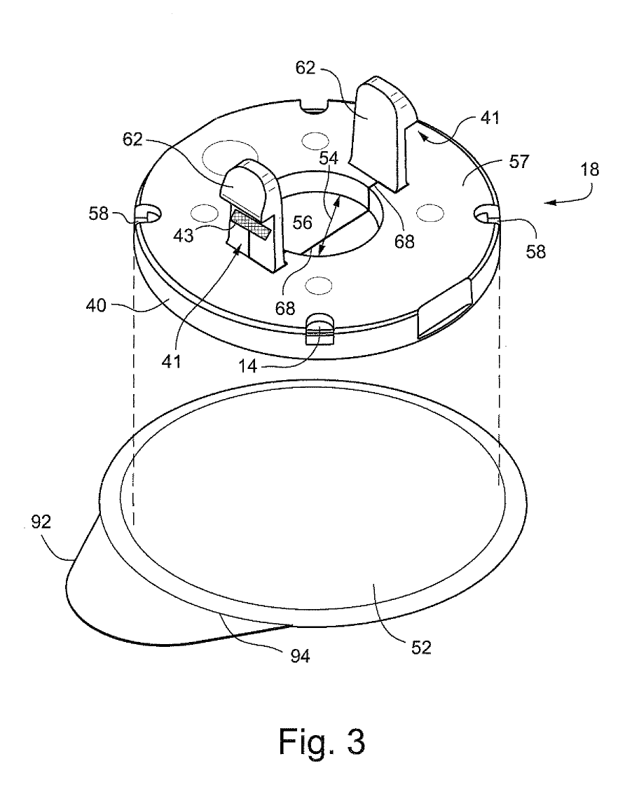

The underside portion of the proximal end 20 provides a grip for the thumb and fingers of one hand to hold the device such as to allow the other hand to insert the cartridge head into the housing and remove the protective lid 52 (FIG. 3) from the head just prior to delivery of the medicament.

To operate the device, the index finger of the other hand is slid into the housing such that the finger is in the ring 27 and the fleshy pad of the finger seats on the conductive pad 31 to provide an electrical path from the finger and into the circuitry of the PCB. The positioning of the cartridge head on the treatment site is relatively easy because the user is moving his index finger to the treatment site and to position the cartridge head on the treatment site. The switch 22 is depressed to activate the device. Electric current flows through the active electrode, medicament matrix and into treatment site. The return path for the electric current is through the patient, conductive surface 31 and to the circuit on the PCB. The current causes medicament to be delivered through the skin at the treatment site of the user. A controller on the PCB may determine the current level and time period to apply the current to the active electrode. Once the medicament has been delivered to the treatment site, the PCB stops delivering current and the device is removed from the treatment site. The cartridge 18 is released from the device by pressing the ejector buttons 26 and is discarded. A new cartridge head may be inserted into the distal end 16 of the housing to apply another dosage of the medicament to a treatment site.

The applicator cartridge 18 is preferably secured to the distal end 16 of the device 10 by a releasable coupling. By providing a two part device 10, e.g., housing 12 and cartridge 18, a fresh applicator cartridge 18 can be applied to the housing 12 for each subsequent use of the device 10. The used applicator cartridge 18 can be ejected or released from the housing by a one-hand operation. A new applicator cartridge head is attached to the device for subsequent treatments.

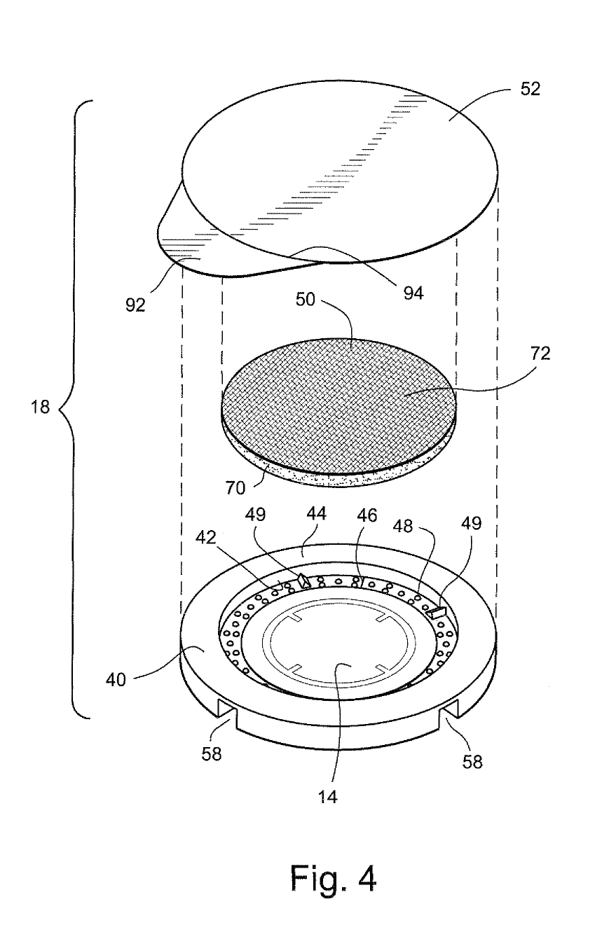

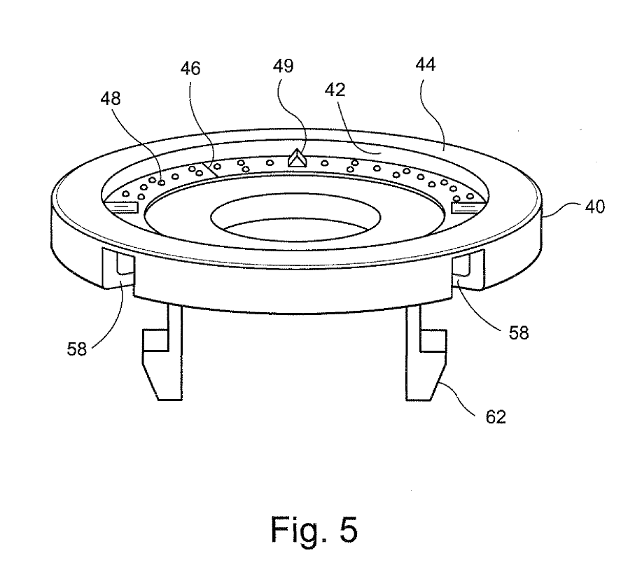

Referring to FIGS. 3 to 5, the applicator 18 generally includes an applicator cartridge head 40, e.g., a disc, having on its back or rear side a locking element 41 for securing the 18 to the device 10. The head 40 of applicator cartridge 18 includes a circular recess 42 on a forward face of the head and defined by a rim 44 of the head 40. An active electrode surface 14 is disposed within the recess 42. The electrode surface may be a separate metal part, a metallized coating on one or more regions of the recess, or a conductive polymer attached to the applicator head.

The base within the rim 44 of the applicator may be shaped to create a concave recess or other profile complementary to the shapes of the electrode and matrix. In a preferred embodiment the perimeter of the active electrode 14 does not extend to the inner wall of the rim 44. An annular surface 46 of the recess remains between the rim and active electrode. It suffices to say that a multitude of electrode sizes, shapes, and materials may be used to provide electrical contact between the applicator recess and the matrix. Also in the prefered embodiment the annular surface 46 includes a plurality of raised projections, e.g., raised dimples 48, ridges 49, or combinations thereof of variable heights projecting from the surface 46. The dimples 48 may be radially spaced from each other across the surface 46.

The electrode 14 may be composed of metal, a metallized polymer or a conductive polymer such as polyaniline, polypyrrole, or a polymer rendered conductive by means of a conductive dopant. The lid 52 over the front face of the head 40 is composed of a polymer laminate with or without a metallic layer. The head 40 may be formed of a polymeric material, such as polypropylene or other polymer inert to the drug formulation in the matrix 50.

To provide an electrical connection between the active electrode 14 of the applicator 18 and the power source, the opposite or second face, e.g., the backside 57, of the head 40, has an opening 54, preferably central to the head 40 through which the backside of the active electrode 14 is exposed. An electrical connection is provided between the backside portion 56 of the electrode 14 and the circuit board when the applicator 18 is secured to the device. The backside 57 of applicator cartridge head 40 also includes one or more openings 58 which also expose a portion of the active electrode 14. The additional exposure of the electrode 14 facilitates the transmission of electrical signals for diagnostic testing during manufacture of the applicator 18.

The backside 57 of applicator cartridge head 40 includes a pair of prongs 62 that form one part of the locking element 41. The distal end 16 of device 10 mounts a pair of flats 64 along an inner surface of the distal end 16. The flats are a second part of the locking element 41. By inserting the prongs within the open inner surface of distal end 16, the prong heads engage the device flats to secure the applicator 18 to the device.

The active electrode 14 makes electrical contact with the circuit board within the distal portion 16. The outer surface of the prongs 62 and an inner cylindrical wall 63 of the distal end 16 may be both electroplated to include a conductive pad 43 (as shown on outer surface of the prong). A conductive line 68 on the surfaces of the head 40 provide an electrical path between the pad 43 on the prongs 62 and the back contact surface 56 of the active electrode. The conductive line 68 may be a bus that includes multiple conductive paths to transmit various signals from different contact points on the active electrode.

When the prongs 62 are inserted in the distal portion 16, the electroplated surfaces 62, 63 abut provide an electrical contact between the circuit board 25 in the housing and the active electrode 14. Further, the surface(s) of the prongs may carry indicia or other markings for lot traceability, medicament identification, prevention of reuse of the applicator, or other information that is “read” by the microprocessor controlled circuitry in the device.

The applicator 18 also includes a matrix 50 which, in a preferred embodiment, is characterized by high void volume and does not interact with the medicament or the electrokinetic delivery of the medicament. The matrix 50 is a carrier supporting the medicament. Acceptable materials for the matrix include but are not limited to variable loft nonwoven and woven materials such as melt-blown, needlepunched, spunbonded, spunlaced or other processed natural fibers, polyolefin, polyester, rayon, nylon, and blends of these, reticulated polyether and polyester polyurethane foams, and silicone foams. Low void volume materials may also be used such as crosslinked hydrogels, interpenetrating polymer networks, scaffolds for immobilizing the active prior to iontophoretic release, highly viscosified formulations, and other matrices that do not rely upon a delivery from a liquid formulation. The matrix may also contain functional components 70 such as reinforcing scrims, networks, and other support structures to facilitate manufacture of the finished product. These layers may also be conductive to ensure homogeneous electrical contact with the drug formulation contained in the matrix. Additionally, the matrix may contain one or more layers carrying arrays of microneedles 72 or other surface features designed to physically penetrate the stratum corneum and promote delivery of medicaments intradermally or transdermally.

A medicament is stored in a head 30 of the cartridge 12. After a lid 50 is removed to expose a porous matrix in the cartridge head, the opened front surface of the cartridge head is applied to the skin of the individual. The porous matrix may be a porous pad, membrane or substrate for the medicament. Acceptable materials for the porous matrix may include but are not limited to variable loft nonwoven and woven materials such as melt-blown, needlepunched, spunbonded, spunlaced or other processed polyolefin, polyester, rayon, nylon, and blends of these, reticulated polyether and polyester polyurethane foams, and silicone foams. Portions of the porous matrix may be conductive to ensure homogeneous electrical contact.

The matrix 50 is attached to the head 40 through mechanical or thermal bonding to a plurality of projections 48, 49. The head 40 may also be constructed with a porous surface to allow penetration and mechanical bonding of matrices such as hydrogels and other materials with low cohesive strength that are cast, injected, thermally formed, or otherwise inserted into the head during assembly. The matrix 50 may be porous so as to support the medicament and a hydration material that carries the medicament into the treatment site upon application of a current by the active electrode. The matrix 50 may by non-porous and carry a medicament that is driven directly by current into the treatment site. In the case of matrices containing thermoplastic fiber content, the matrix may be ultrasonically attached to the head 40 at one or more locations determined by contact with the array of raised projections, e.g., dimples 48 and bars 49. Such projections are designed to focus the ultrasonic energy within the matrix and limit the extent of co-melting of the projections and the thermoplastic components of the matrix. This conserves the absorptivity of the matrix and improves the cosmetic appearance of the matrix-to-base bond sites. It is understood that ultrasonic bonding of the matrix to the base of the cartridge head 40 may be adjusted to accommodate the size and shape of the electrode surface. In a preferred embodiment the projections 48, 49, during ultrasonic welding, focus and direct the ultrasonic energy such that only the margin of the matrix 50 is secured to the margin 46 at the bottom of the recess 42 leaving the central portion of the matrix unattached to the underlying active electrode 14.

The margin 46 is an annular surface on the base which is in the recess 42 of the cartridge head 40. The margin 46 is concentric with respect to an outer rim 44 which is an annular surface extending around the periphery of the recess 42. The polymeric materials forming the cartridge head 40 and matrix 50 may include copolymers and homopolymers. When the medicament or a medicament and hydration carrier for the medicament is disposed in the matrix 50, particularly the non-woven material layers 70, contact with the active electrode 14 is between the electrode 14 and the medicament or the medicament and its carrier.

To maintain the applicator 18 in a sealed condition prior to use, there is provided a lid 52 which seals about the outer rim 44 of the head 40. The lid 52 is formed of a plurality of layers. For example, as illustrated in FIG. 7, the bottom layer is a heat seal layer 82 followed by a plastic backing or polypropylene layer 84. A vapor barrier layer 86 lies intermediate the opposite faces of the lid followed by a foam layer 88 and a top polyester (Mylar®) layer 90. The vapor barrier layer may be comprised of metal foil, metalized polymer, or coating that prevents escape of volatiles from the applicator when the lid is sealed. In general, the lid 52 may be composed of any material providing a hermetic seal over the drug formulation and pad. The lid 52 additionally has a tab 92 which preferably projects laterally from one side of the lid and beyond the rim 44 of the head.

The tab 92 may be one or more projections from the otherwise circular lid. Alternatively, the tab may be a ring of the lid that projects laterally beyond the head 40 of the applicator. The tab 92 and the lid 52 are formed of the same layered material. The lid is preferably induction sealed about the outer rim 44 of the front face of the cartridge head 40. Induction sealing uses an RF field to create heat in a metallic layer, i.e., the middle layer 86 which in turn melts the polymer layer to effect a heat seal with the head 40 of the applicator cartridge 18. To prevent the tab 92 from acting as a heat sink which would cause the seal in the vicinity of the tab to lag behind the heat sealing of the lid to the head 40 in areas of the head remote from the tab, a discontinuity is provided between or at the interface of the tab and the lid. The discontinuity may be in the form of a kiss cut 94 which interrupts the thermal path to the tab from the lid when heat is applied. At the interface, at least the foil layer is cut, for example not less than 50-70% of its length along the tab/lid interface to prevent the tab from acting as a heat sink. If a tab formed of plastic or other insulator is used, there may be no need for a foil cut.

The kiss cut 94 may not be necessary if the lid is secured to the head 40 by means other than induction heating, e.g., an adhesive or heat sealing. Also, the tab 92 need not project laterally as illustrated. For example, a central pull tab, e.g., in a semi-circular form, may be used to remove the lid from the head 40.

To utilize the electrokinetic delivery device hereof, the device 10 is switched on. The individual may then apply a conductivity enhancer (towelette, hydrogel, or equivalent) to the index finger, insert the finger into the device 10, remove the protective lid, and apply the applicator via the device to the face. The electrokinetic current flows from the device active electrode 14 through the face thus delivering the active into the skin and returns through the finger and into the counter electrode ring 60.

The applicator 18 is provided with the matrix 50 prefilled with the medicament or the medicament and an electrical carrier therefor. Alternatively, the medicament or the medicament and electrical carrier therefor can be applied manually to the face of the matrix 50 upon removal of the lid. The medicament or medicament and carrier are carried by the applicator head in either method of application.

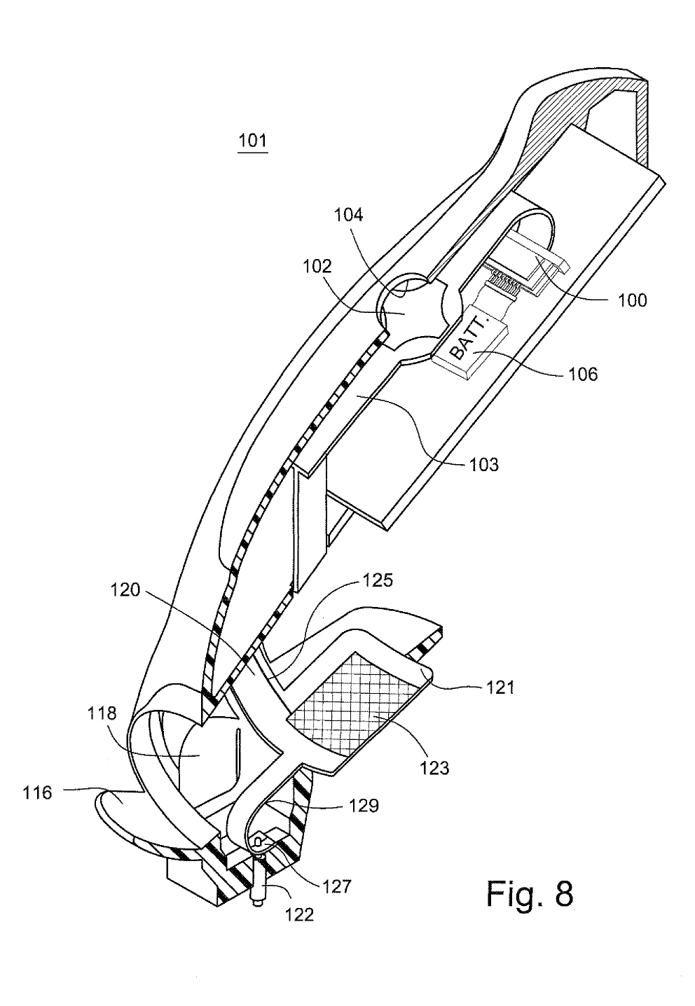

FIG. 8 is a perspective view of an alternative housing 101 having a flexible electrical circuit 103 (flex circuit) disposed within the housing. The flex circuit 103 includes a printed circuit board (PCB) board 100, an on-off switch 102 accessible through an opening 104 in the housing 101 and activated by a button (not shown) accessible at the top of the housing. The flex circuit is connected to the circuitry of the PCB. The flex circuit includes an arcuate portion 120 that forms a partial ring and seat 121 for a finger inserted into the housing. The arcuate portion of the flex circuit has a surface to receive the finger and on that surface is a counter electrode pad 123 to receive the fleshy pad of the finger in the housing. A conductive line 125 extends from the counter electrode pad 123 to the PCB to provide an electrical connection between the counter electrode and the circuits on the PCB. On the opposite side of the flex circuit is another conductive line 127 that provides an electrical connection between the PCB and a pin 122 that is biased against the backside of the active electrode when a cartridge is inserted in the housing. The pin 122 and conductive line 127 establish an electrical connection between the active electrode and the PCB. The pin is biased towards the active electrode by a spring portion 129 of the flex circuit.

Flexible circuit designs, e.g., circuit 120, are known in the industry for connecting a collection of individual components in a cost-effective electronic package. Electrical circuitry is provided to initiate treatment, i.e., actuating the current to apply the medicament upon locating the device with the cartridge in contact with the treatment site. To accomplish this, switch contacts (not shown) may be placed in the circuit, such as in conjunction with the engagement features of the cartridge prongs 62 or with the spring pin 122.

Flexible circuit designs, e.g., circuit 120, are known in the industry for connecting a collection of individual components in a cost-effective electronic package. Electrical circuitry is provided to initiate treatment, i.e., actuating the current to apply the medicament upon locating the device with the cartridge in contact with the treatment site. To accomplish this, a switch contacts (not shown) may be placed in the flex circuit, such as in conjunction with the counter electrode 123 on the finger seat 121 and/or with the pin 122 and spring 129.

When the individual's finger is placed in the seat 121, the contact actuates the circuit when the individual presses the device i.e., the cartridge against the treatment site. The act of pressing a finger on the seat 121 and/or pressing the cartridge head against the treatment site creates an electrical connection between the power source in the housing and the active electrode in the cartridge. Conversely, releasing pressure on the finger seat 121 or removing the head from the treatment site, interrupts the electrical circuit between the active electrode and the power source. This interruption is sensed and used by a controller in the housing to reduce the open circuit voltage as well. Use of this “push to treat” mechanism also reduces costs by eliminating the need to monitor current as an indicator of facial contact and potentially enabling a smaller pin count, and a less sophisticated and cheaper microcontroller. Also, this reduces patient discomfort by using patient action upon application and removal of the treatment device relative to the treatment site to control voltage and current.

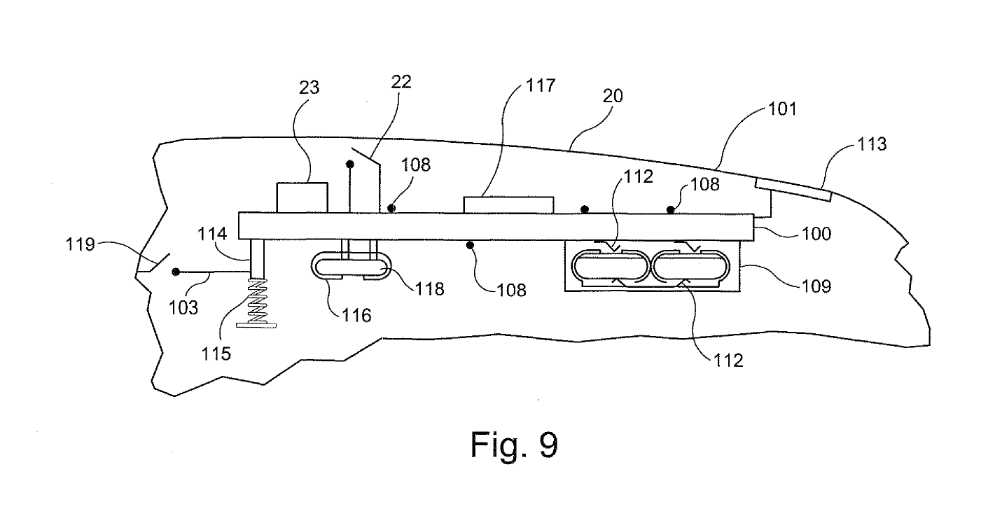

FIG. 9 is a schematic side view of the internal electrical circuit in the housing 101. The circuit includes the PCB (printed circuit board) 100 that is supported by posts integrally molded with the housing. A battery well 109 contains batteries 110 that are biased by spring connectors 112 against terminals in the PCB and provide electrical contacts between the batteries and the PCB. A contact pin 114 provides a connection between the PCB and the flex circuit 103. A spring 115 biases the contact pin 114 against the PCB. A audible alarm 116 may be formed of a piezo-electric device that vibrates when activated by a controller 117 on the PCB. The alarm 116 may be supported by spring contacts in the housing and biased against the PCB. For example, the piezo of the alarm 116 slides into a resident cavity 118 in the housing. When the PCB is installed, spring contacts mate with the piezo alarm, light pipe 23, and batteries to provide electrical contacts to the PCB. The microcontroller 117 is mounted directly on the PCB.

The switch 22 may be a stamped metal contact switch or other sensing mechanism in the cartridge prong area. The switch is used to power up the device and constitutes a cartridge (ON/OFF) switch. This power up switch can be incorporated with the “push to treat” contact turning the device on and off in its entirety by applying the device to the face. This enables a cost reduction by reducing the number of small input output ports for the microcontroller making power connections directly with the battery.

The microcontroller 117 or processor provides intelligent control for the operation of the device. The controller may sense a unique electric identifier, e.g., encrypted, on the cartridge head and/or prongs and thereby confirm that the cartridge head is authorized for use with the device. Further the controller may prevent reuse of a cartridge head by storing information when a uniquely identified head is used and/or by coding the head upon use, e.g., by fusing an electrical contact on the head and/or prongs to signify that the head has been used. The controller may also identify a particular drug formulation in the matrix based on an electric code carried by the cartridge head and/or prongs. Using the drug formulation, the controller may setup the device for imparting electrical current levels and current application times that is predetermined for the identified drug formulation. In this way, a single applicator device may be automatically adapted to control the iontophoresis dose of various drug formulations which have different prescribed current levels and current application times.

The electrokinetic current provided by the power supply and governed by the controller 117 drives a therapy indicator 113, e.g., a liquid crystal display (LCD) or a light emitting diode (LED) 23. The indicator minimizes the circuitry necessary to deliver therapy (especially if the indicator is embodied in the light 23) and provides appropriate feedback to the individual user regarding dosage of the medicament. A possible feedback element that can be provided by the light is providing an indication to the user that therapy is being delivered. The present invention reduces circuit complexity by placing an LED 23 in line with the therapeutic current flow so that the LED is illuminated when current is applied to the active electrode and dimmed when current is not applied to the active electrode. For example, as the therapeutic current flows the LED 23 glows. The LED will continue to indicate therapeutic current flow until the device is removed from the treatment site and current no longer flows.

The electrokinetic current provided by the power supply and governed by the controller 117 may drives a therapy indicator 113, e.g., a liquid crystal display (LCD) or a light such as the light pipe 23. The indicator minimizes the circuitry necessary to deliver therapy (especially if the indicator is embodied in the light pipe 23) and provides appropriate feedback to the individual user regarding dosage of the medicament. A possible feedback element that can be provided by the light pipe is providing an indication to the user that therapy is being delivered. The present invention reduces circuit complexity by placing an LED 23 in line with the therapeutic current flow so that the light pipe 23 is illuminated when current is applied to the active electrode and dimmed when current is not applied to the active electrode. For example, as the therapeutic current flows the LED 23 glows. The LED will continue to indicate therapeutic current flow until the device is removed from the treatment site and current no longer flows.

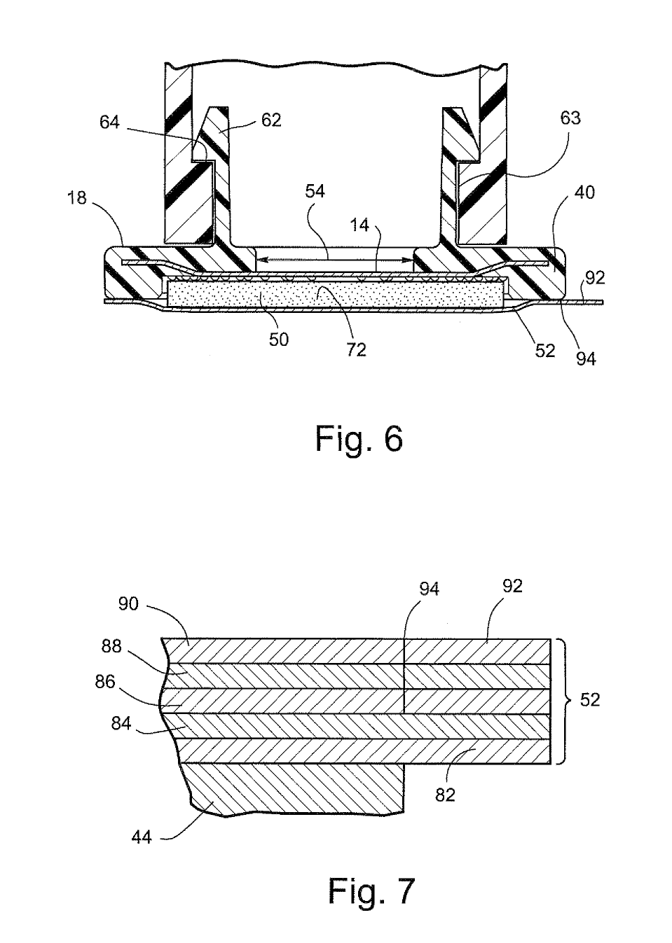

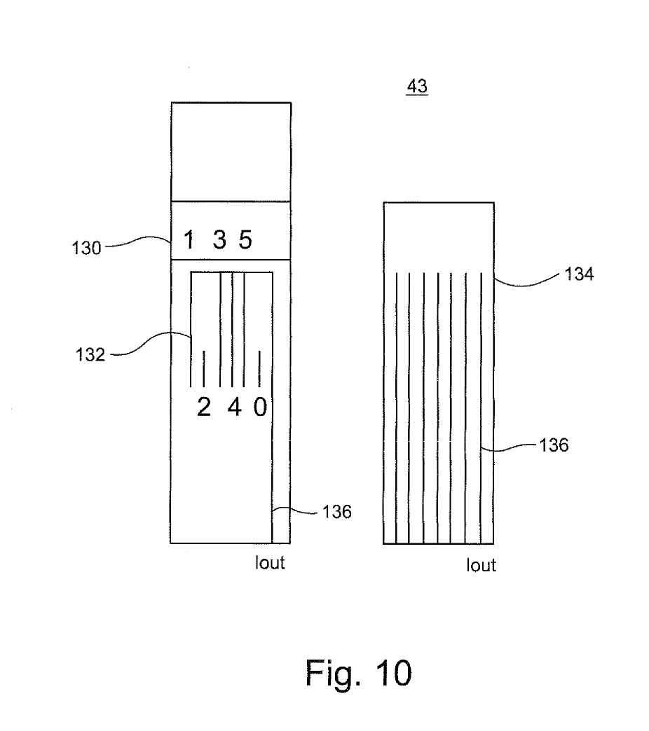

FIG. 10 show exemplary contact pads 43 for the connection between the cartridge head 40 and the housing 12. The contact pads may be mounted on an outer surfaces of one of the prongs and an inside surface 63 of the distal end of the housing where the prongs are received. (see FIG. 6). The contact pad on the prong may be an encoded series of elecro-plated contacts 130 that identifies the cartridge head. For example, the coding may be vertical conductive lines 132 on the pad and arranged in parallel. The lines 132 may each be associated with a particular reference number, e.g., 1 to 5. By encoding the contact 130 with select lines, e.g., 1, 3 and 5, the contact is coded with a prescribed reference indicator. The opposing contact pad 134 on the inside surface of the housing is a generic contact that establishes electrical contact with all of the possible selected lines on the contact pad of the cartridge head. The microcontroller is in electrical communication with the contact pad 134 and senses the selected lines 132 of the contact pad on the prong(s) of the cartridge head. Based on the detected lines 132 on pad 134 of the cartridge head, the controller determines if the cartridge is suitable for the device, whether the cartridge has been previously used in the device and/or the appropriate current levels and current application period for the cartridge. Further, the opposing contact pads 130,134 both include a line-out (lout) power lines 136 that provide an electrical contact for current to the active electrode. The use of the lout lines reduces the risk that a user will be burned by inadvertently applying current to his skin using the device without a cartridge because the lout line is internal to the device and not easily put in contact with skin. The lout lines 136 also avoid the need for a pin 122 and interlock switch.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

We claim:

1. An applicator for use with a device for electrokinetically delivering a medicament to a treatment site comprising:
an applicator head including an active electrode;
a matrix support surface of said applicator head extending beyond said active electrode having a plurality of spaced projections;
a matrix attached to the matrix support surface, and the active electrode adjacent the matrix, and
a medicament or a medicament and an electrically conductive carrier carried by said matrix in electrical contact with said active electrode.

2. The applicator according to claim 1 wherein the matrix and applicator head are ultrasonically welded together.

3. The applicator according to claim 1 further comprising a releasable lid attached to a lid support surface of the applicator head, wherein the matrix support surface is recessed in said head with respect to the lid support surface.

4. The applicator according to claim 3 wherein the lid support surface is an annular rim attached to the applicator head and the matrix support surface is an annular margin concentric with the margin.

5. The applicator according to claim 1 wherein the projections of the matrix support surface are formed of a deformable material that deforms to bond the matrix to the matrix support surface.

6. The applicator according to claim 1 wherein the matrix support surface is recessed from an outer face of the applicator head.

7. The applicator according to claim 1 wherein the matrix has a central portion not bonded to the matrix support surface.

8. The applicator according to claim 1 wherein the applicator head and matrix are formed of a polymer material.

9. The applicator according to claim 1 wherein the matrix is secured to the applicator head solely about the matrix support surface, and a central portion of the matrix is unsecured to and in electrical contact with the active electrode.

10. The applicator according to claim 1 wherein said matrix includes one or more layers containing microprojections used to penetrate the stratum-corneum layer of the skin.

11. The applicator according to claim 1 including a lid overlying the matrix and releasably secured to the applicator head.

12. The applicator according to claim 11 wherein said lid comprises layers of different materials, one of said layers being formed of a metallic material, and at least a portion of an interface between the metallic material of the tab and metallic material of the lid having a discontinuity.

13. The applicator according to claim 11 wherein the lid includes a tab projecting laterally from said lid.

14. The applicator according to claim 13 wherein a discontinuity between the tab and lid interrupts at least in part a thermal path between said tab and said lid at least along said metallic material.

15. The applicator according to claim 11 wherein said lid is heat sealed to the applicator head and the lid includes a tab for removing the lid from the applicator head.

16. The applicator according to claim 1 wherein said active electrode includes at least one electrode portion thereof exposed through an opening in the applicator head for electrical connection with a power source carried by the device.

17. The applicator according to claim 16 wherein the one electrode portion is exposed through a central portion of the applicator head.

18. The applicator according to claim 16 wherein said matrix is in contact with the active electrode on a side of the electrode opposite to the one electrode portion.

19. An applicator for use with a device for electrokinetically delivering a medicament to a treatment site comprising:
an applicator head having an active electrode and a matrix overlying the active electrode;
a medicament or a medicament and an electrically conductive carrier carried by said matrix and in electrical contact with said electrode, and
a lid overlying the matrix on a side of the matrix remote from said electrode and releasably secured to the applicator head.

20. The applicator according to claim 19 wherein said lid includes a tab and a discontinuity between the tab and a remainder of the lid.

21. The applicator according to claim 20 wherein said lid is induction welded to the applicator head and said discontinuity interrupts at least in part a thermal path between said tab and said lid.

22. The applicator according to claim 19 wherein lid includes a heat seal layer for sealing the lid to the applicator, a metallic foil layer and a polyester layer.

23. The applicator according to claim 20 wherein said discontinuity is in a foil layer of the lid.

24. The applicator according to claim 19 wherein said active electrode includes a contact surface opposite to a surface in contact with the matrix and the contact surface is exposed for electrical connection with a power source carried by the device.

25. The applicator according to claim 19 wherein the one electrode portion is exposed through said first face along a central portion of the applicator head, said active electrode having a second portion exposed through said first face and spaced from said first portion.

26. An applicator device for electrokinetically delivering a medicament to a treatment site comprising:
a housing including a power supply and an electronic circuit for providing a current to deliver the medicament, wherein the housing includes an aperture to receive a finger;
an applicator head including an active electrode and a matrix overlying said electrode;
a medicament or a medicament and an electrically conductive carrier carried by said matrix;
a margin of said cartridge beyond said active electrode and a matrix being secured to one another,
said active electrode having a first portion thereof exposed through said applicator head and a second portion, opposite to the first, in electrical contact with the matrix, and
a counter electrode in the housing mounted on a surface adapted to contact the finger and the counter electrode in electrical communication with the electronic circuit.

27. The applicator device according to claim 26 wherein said active electrode has a second portion of the electrode exposed through the first face of the applicator head and at a location spaced from the first electrode portion.

28. The applicator device according to claim 26 wherein the counter electrode is on an inner surface of a ring in the housing.

29. The applicator device according to claim 26 wherein the counter electrode is on first surface of flexible circuit in the housing and an opposite surface of the flexible circuit include an electrical path for the active electrode.

30. The applicator according to claim 29 wherein the flex circuit includes a spring which biases a connector pin against the active electrode.

31. An applicator for use with a device for electrokinetically delivering a medicament to a treatment site comprising:
an applicator head having a recess, an active electrode in said recess and a matrix in said recess overlying said active electrode;
a medicament or a medicament and an electrically conductive carrier carried by said matrix;
a margin of said applicator head within said recess, said matrix being secured to the margin, and
said active electrode including an electrical contact exposed through a side of said applicator head opposite to the recess.

32. The applicator according to claim 31 wherein the matrix has a central portion inward of the matrix margin.

33. The applicator according to claim 31 wherein said applicator head and said matrix are formed of polymer materials, said margin including a plurality of raised projections facilitating ultrasonic welding of said matrix and said head to one another.

34. The applicator according to claim 31 wherein the margin includes projections that bond to the matrix.

35. The applicator according to claim 34 wherein the projections are dimples.

36. The applicator according to claim 31 wherein one or more of the surfaces of the applicator head are porous.

37. The applicator according to claim 31 wherein one or more of the surfaces of the applicator head are non-porous.

38. The applicator according to claim 31 wherein the margin is ultrasonically bonded to the matrix.

39. An applicator device for electrokinetically delivering a medicament to a treatment site comprising:
a housing including a power supply and an electronic circuit providing current for delivering the medicament;
a cartridge head having an active electrode and a matrix overlying the active electrode, wherein the active electrode is in electrical communication with power supply and electronic circuit when the head is inserted into the housing;
a medicament or a medicament and an electrically conductive carrier carried by said matrix and in electrical contact with said active electrode, and
a locking element on the cartridge head and releaseably engaged with the housing, the locking element having a first conductive contact surface abutting a second conductive contact surface in the housing,
wherein the first conductive contact surface is in a conductive path with the active electrode and the second conductive contact surface is in a conductive path with the power supply and electronic circuit, and further the abutment of the first conductive surface and the second conductive surface establishes a electrical connection between the surfaces.

40. An applicator device as in claim 39 wherein the first conductive contact surface is encoded and the second conductive contact surface has electrical contacts to sense the coding on the first conductive contact surface.

41. An applicator device as in claim 40 wherein the first conductive contact surface is encoded by having one or more of and not all of selected conductive lines.

42. An applicator device as in claim 1 wherein the first conductive contact surface and the second conductive contact surface each have conductive power lines which abut when the cartridge head is locked into the housing.
